SEQUENCE LISTING (1 sequence)

SEQ ID NO: 1 - protein, 137 aa, Escherichia coli
MIWKRHLTLDELNATSDNTMVAHLGIVYTRLGDDVLEAEMPVDTRTHQPFGLLHGGASAALAETLGSMAGFMMTRDGQCVVGTELNATHHRPVSEGKVRGVCQPLHLGRQNQSWEIVVFDEQGRRCCTCRLGTAVLG

(19) United States
(12) Patent Application Publication
(10) Pub. No.: US 2024/0360486 A1
(43) Pub. Date: Oct. 31, 2024

(54) METHODS AND COMPOSITIONS FOR THE PRODUCTION OF MALONATE

(71) Applicant: THE REGENTS OF THE UNIVERSITY OF CALIFORNIA, Oakland, CA (US)

(72) Inventors: Deepika AWASTHI, Emeryville, CA (US); Shubhasish GOSWAMI, Berkeley, CA (US); Steven W. SINGER, Oakland, CA (US)

(21) Appl. No.: 18/649,779

(22) Filed: Apr. 29, 2024

Related U.S. Application Data
(60) Provisional application No. 63/498,763, filed on Apr. 27, 2023.

(51) Int. Cl. C12P 7/46; C12N 1/20; C12N 9/16; C12R 1/01

(52) U.S. Cl. CPC C12P 7/46; C12N 1/205; C12N 9/16; C12Y 301/02018; C12R 2001/01

(57) ABSTRACT

The present invention provides for a genetically modified host cell comprising one or more enzymes described herein, wherein the genetically modified host cell is capable of producing malonate from methane.

### CROSS-REFERENCE TO RELATED APPLICATIONS

This application claims the priority benefit of U.S. Provisional Application No. 63/498,763, filed Apr. 27, 2023, which is hereby incorporated by reference in its entirety.

### STATEMENT OF GOVERNMENTAL SUPPORT

The invention described and claimed herein was made utilizing funds supplied by the U.S. Department of Energy under Contract No. DE-AC02-05CH11231. The government has certain rights in this invention.

### REFERENCE TO SEQUENCE LISTING

The instant application contains a Sequence Listing which has been submitted electronically in XML format and is hereby incorporated by reference in its entirety. Said XML copy, created on Apr. 29, 2024, is named “2022-098-02 Sequence Listing.xml” and is 3 kilobytes in size.

### FIELD OF THE INVENTION

The present invention is in the field of production of malonate.

### BACKGROUND OF THE INVENTION

Malonic acid (MA) is also listed as one of the top 30 chemicals which can be produced from renewable sources, as defined by the U.S. Department of Energy. Malonic acid is a specialty chemical for a number of manufacturing processes. There is a $1 billion in annual derivative specialty chemical demand for malonic acid. Malonic acid and its esters have historically been produced from cyanide and chloroacetic acid process, which involves toxic, health and environmentally hazardous chemicals. Bioconversion can provide identical performance with environment friendly process. Currently, microbial production involves converting sugar into malonic acid.

| Microbe | Precursor | Description | Titer | | Fermentation | Refs |
|---|---|---|---|---|---|---|
| Escherichia | B-Alanine | +ppc, aspA, and ynel from | 0.45 | g/L | Fed batch | Song et al. |
| coli | | E. coli; +panD from | 3.6 | g/L | Shake flask | (2016) |
| | | Corynebacterium | | | | |
| | | glutamicum; +pa4123 from | | | | |
| | | Pseudomonas aeruginosa; | | | | |
| | | ΔydfG | | | | |
| E. coli | Acyl-CoA | +ehd3 mutant from | 82.3 | g/L | Shake flask | Dietrich et |
| | | Saccaromyces cerevisiae | | | | al. (2017) |
| Pischia | Acyl-CoA | +ehd3 mutant from | 76 | g/L | Shake flask | Dietrich et |
| kudriavzevii | | S. cerevisiae; +Anmae1 from | | | | al. (2017) |
| coli | | Aspergillus niger | | | | |
| Myceliophthora | Oxaloacetate | +mdc from Ogataea | 42.5 | g/L | Shake flask | This study |
| thermophila | | parapolymorpha; ynel from | | | | |
| | | E. coli; +glt-1 from | | | | |
| | | Neurospora crassa | | | | |

Note.
“+” represents overexpression of target gene;
“Δ” represents disruption of target gene.

### SUMMARY OF THE INVENTION

The present invention provides for a genetically modified host cell comprising one or more enzymes described herein, wherein the genetically modified host cell is capable of producing malonate from methane. In some embodiments, the genetically modified host cell comprises one or more modifications described in U.S. Provisional Patent Application Ser. No. 63/346,788, which is incorporated by reference in its entirety. The present invention provides for a genetically modified host cell converting CH₄ and CO₂ into malonate. CH₄ and CO₂ are greenhouse gases (GHG).

The present invention provides for a genetically modified host cell is Methylomicrobium species cell comprising a heterologous acyl-CoA hydrolase, or homologous enzyme thereof, having the enzymativ activity to convert malonyl-CoA into malonate. In some embodiments, the genetically modified host cell comprises a nucleic acid encoding the heterologous acyl-CoA hydrolase operatively linked to a promoter capable of expressing the heterologous acyl-CoA hydrolase, or homologous enzyme thereof, in the genetically modified host cell.

In some embodiments, the Methylomicrobium species cell is Methylomicrobium alcaliphilum cell. In some embodiments, the Methylomicrobium alcaliphilum cell is Methylomicrobium alcaliphilum strain DSM19304. In some embodiments, the Methylomicrobium alcaliphilum strain DSM19304 is DASS strain. In some embodiments, the acyl-CoA hydrolase is a short-chain acyl-CoA hydrolase. In some embodiments, the acyl-CoA hydrolase is a short-chain acyl-CoA hydrolase. In some embodiments, the acyl-CoA hydrolase is an Escherichia coli (E. coli) POA8Z3, E. coli POA8Y8, Haemophilus influenzae Rd KW20 YBGC, H. influenzae YciA, or Mesocricetus auratus Acot9, or a wild-type or homologous enzyme. In some embodiments, the acyl-CoA hydrolase comprises at least one or more, or all, of the conserved amino acid residues compared to any two of any of the following: E. coli POA8Z8, Salmonella typhimurium proofreading thiosterase EntH, Salmonella schwarzengrund proofreading thiosterase EntH, E. coli proofreading thiosterase EntH, Citrobacter koseri proofreading thiosterase EntH, Salmonella paratyphi A proofreading thiosterase EntH, Salmonella paratyphi B proofreading thiosterase EntH, Shigella flexneri proofreading thiosterase EntH, Shigella dysenteriae proofreading thiosterase EntH, and Shigella sonnei proofreading thiosterase EntH. All of these EntH are identified in the webpage for: uniprot.org/uniprotkb/POA8Y8/entry, and their respective amino acid sequences are publicly available. In some embodiments, the acyl-CoA hydrolase comprises one or more of the following amino acid residues corresponding to SEQ ID NO: 1 is substituted with a different amino acid: P49, G51, A60, G66, H89, H90, P92, and I116. In some embodiments, the amino acid residue(s) is substituted with an amino acid residue with a bulky sidechain. In some embodiments, the amino acid residue(s) is substituted with a phenylalanine.

In some embodiments, one or more enzymes comprises PmoC, MxaF, Hps, Hpi, Pfk, Fbk, Pgk, Pyk, Pdh, AccA, and/or malonyl Co-A hydrolase. Each of the one or more enzymes are either endogenous or native to the host cell, or introduced into the host cell, such as one or more nucleic acids encoding the one or more enzymes, wherein each is operatively linked to a promoter, introduced in the host cell. In some embodiments, each nucleic acid is part of a vector capable of stably residing in the host cell, or stably integrated into the host cell's genome.

The present invention provides for a method for producing a malonate, the method comprising: (a) providing a genetically modified host cell of the present invention, capable of expression in the host cell in a growth or culture medium comprising methane; (b) growing or culturing the host cell such that a malonate is produced; and (c) optionally recovering the malonate from the host cell or from the growth or culture medium. In some embodiments, the host cell is a methanotroph. In some embodiments, the growing or culturing step results in the host cell or methanotroph producing more malonate than the host cell or methanotroph in its unmodified form. In some embodiments, the growing or culturing step results in the host cell or methanotroph producing malonate at a concentration equal to or less than about 1.0 g/L, 1.5 g/L, 2.0 g/L, 2.5 g/L, 3.0 g/L, 3.5 g/L, 4.0 g/L, 4.5 g/L, or 5.0 g/L, or within a range of any two preceding values. In some embodiments, the growing or culturing step results in the host cell or methanotroph producing malonate at a concentration within a range of about any two titer or yield values described herein.

The present invention provides for a growth or culture medium comprising a genetically modified host cell of the present invention. In some embodiments, methane is the sole carbon source in the growth or culture medium.

In some embodiments, the host cell comprises a nucleic acid encoding one or more of the one or more enzymes, or homologous enzyme(s) thereof, operatively linked to one or more promoters capable of expressing the one or more enzymes, or homologous enzyme(s) thereof, in the host cell. In some embodiments, the one or more enzymes are encoded on a vector, plasmid, or plasmid-based system. In some embodiments, the nucleic acid encoding one or more enzymes is codon optimized for the host cell. In some embodiments, the host cell in the unmodified form does not produce malonate.

In some embodiments, the host cell is a methanotroph. A methanotroph is a bacterium that is able to use methane as a sole carbon source for growth. In some embodiments, the methanotroph is a Gram-negative, haloalkaliphilic, and/or obligate methanotroph. In some embodiments, the host cell is a methanotroph is a Methylococcus or Methylotuvimicrobium cell. In some embodiments, the Methylotuvimicrobium cell is a Methylotuvimicrobium buryatense or Methylotuvimicrobium alcaliphilum (syn. Methylomicrobium alcaliphilum). M. alcaliphilum is a Gram-negative, haloalkaliphilic, obligate methanotroph that has been sequenced and for which basic genetic tools for engineering and gene expression have been developed.

The present invention provides for a methanotroph comprising one or more, or all, of the mutations indicated in the attached Appendix. In some embodiments, the host cell or methanotroph comprises one or more, or all, of the mutations indicated in the attached Appendix. In some embodiments, the host cell or methanotroph comprises mutations in the genes of one or more, or all, of the following: IS3 family transposase (gene-MEAL Z_RS03460, gene-MEALZ RS08615, gene-MEALZ_RS11580); autotransporter outer membrane beta-barrel domain-containing protein (gene-MEALZ_RS22650); Crp/Fnr family transcriptional regulator (gene-MEALZ_RS12360); multicopper oxidase domain-containing protein (gene-MEALZ_RS14455); type IV pilus secretin PilQ (pilQ; gene-MEALZ_RS15795); and, FAD-dependent oxidoreductase/hypothetical protein/NAD (P)/FAD-dependent oxidoreductase (gene-MEALZ_RS18045). In some embodiments, the host cell or methanotroph comprises endogenous genes of one or more, or all, of the genes/proteins indicated in the Appendix, or for IS3 family transposase; autotransporter outer membrane beta-barrel domain-containing protein; Crp/Fnr family transcriptional regulator; multicopper oxidase domain-containing protein; type IV pilus secretin PilQ; and, FAD-dependent oxidoreductase/hypothetical protein/NAD (P)/FAD-dependent oxidoreductase.

In some embodiments, the host cell or methanotroph is not a natural malonate producer. In some embodiments, the host cell or methanotroph in its unmodified form is sensitive, or unable to grow, in a medium having malonate at a concentration equal to or more than about 0.5 g/L, 0.6 g/L, 0.7 g/L, 0.8 g/L, 0.9 g/L, 1.0 g/L, or 1.5 g/L, or within a range of any two preceding values. In some embodiments, the host cell or methanotroph when modified is resistant, or able to grow, in a medium having malonate at a concentration equal to or less than about 1.0 g/L, 1.5 g/L, 2.0 g/L, 2.5 g/L, 3.0 g/L, 3.5 g/L, 4.0 g/L, 4.5 g/L, or 5.0 g/L, or within a range of any two preceding values. In some embodiments, the host cell or methanotroph when modified is capable of producing more malonate and/or fatty acids than the host cell or methanotroph in its unmodified form. In some embodiments, the methanotroph can produce more malonate and/or fatty acids using methane as the sole carbon source.

Malonic acid qualifies the top-10 biochemical target on DOE's list. Malonate has an existing multi-billion-dollar market in industries such as polyester textiles and apparels, automobile coatings, polymers, flavors, fragrances and vitamin synthesis. Currently, it is commercially produced by petroleum sources and by one known biological route-sugar fermentation by yeast (Lygos). Biologically, malonic acid is produced as a side product of cellular metabolism in higher eukaryotes like plants and animals. A synthetic route was engineered to produce malonate in yeast (unicellular eukaryotes) which has been commercialized (Lygos). So far, no known bacterial (prokaryote) routes have been characterized for malonic acid as the chief product of interest. Thus, in this invention, which is at idea and proof of concept development stage, we are:
1) Using Methylomicrobium alcaliphilum strain DASS (ROI-provisional IP: 2019-167)-a prokaryote bacteria as platform host, which grows on methane as the carbon source in a defined mineral salts media. Strain DASS is described in U.S. Patent Application Publication No. 2023/0399666 (Awasthi et al, “Methods and compositions for the production of rhamnolipid”), hereby incorporated by reference.
2) Developing CRISPRI (dCas9) technique to identify genetic targets in M. alcaliphilum DASS genome (U.S. Provisional Patent Application Ser. No. 63/346,788, which is incorporated by reference in its entirety), interference/deletion of which will increase malonyl-CoA production (precursor to malonate and fatty acids)
3) Screening short-chain acyl-CoA hydrolases for malonyl-CoA to malonate bioconversion. Enzyme candidates include: Escherichia coli (E. coli) YBGC (uniport ID: POA8Z3), E. coli YciA (EcYciA) (uniport ID: POA8Y8), Haemophilus influenzae Rd KW20 YBGC, H. influenzae YciA, Mesocricetus auratus Acot9.
4) Performing Site-directed mutagenesis on candidate enzymes (listed in 3) to achieve specific malonyl-CoA hydrolase activity.

Development of an economical and sustainable bioprocess, utilizing waste and a greenhouse gas-methane as the primary feedstock, is speculated to reduce our dependence on petrochemical based, current malonate producing processes.

Methanotroph, Methylomicrobium alcaliphilum DASS (obligate methane assimilating gram-negative bacteria) is derivative of strain DSM19304. Strain DASS was developed in a previous study (disclosed in U.S. Patent Application Publication No. 2023/0399666) by us and innately secretes high fatty acids and produces more malonyl-CoA than its parent DSM19304 (Awasthi et. al, 2022). Therefore, M. alcaliphilum is employed as the base strain to perform screening, engineering and expression of recombinant short-chain acyl-CoA hydrolases and subsequent bioengineering targeting recombinant malonate bioproduction (E. coli YBGC (uniport ID: POA8Z3), E. coli YciA (EcYciA) (uniport ID: POA8Y8), Haemophilus influenzae Rd KW20 YBGC, H. influenzae YciA, Mesocricetus auratus Acot9). The novelty of this work is, (a) demonstrating malonate production using methane (CH₄) as the primary carbon source; (b) engineering new enzyme(s) for malonate biosynthesis; (c) incorporating Carbon dioxide (CO₂) as the secondary carbon source to increase product yields, thus per mol of (C3) malonate will be produced with 2 mol (C1) CH₄ and 1 mol CO₂. Thus, establishing a unique and new bacterial pathway for high titer malonate production.

Milestone: Proof of concept demonstration of malonate production in M. alcaliphilum DASS.

Future work: identify gene knockout targets to increase malonyl-CoA pool for high titer malonate biosynthesis (U.S. Provisional Patent Application Ser. No. 63/346,788, which is incorporated by reference in its entirety). Process optimization to achieve high titer, yield and rate of malonate biosynthesis.

Current companies with existing methane bioprocess platform, can use the M. alcaliphilum strains for directly synthesizing malonate. Shell and Exxon, oil companies are also looking at R&D on natural gas conversion to products. Natural gas is mainly a mixture of methane (about 95%) with other organic gases and M. alcaliphilum strains can be a beneficial biocatalyst in establishing methane conversion technology to commodity chemicals.

The use of malonate is not limited to one specific area and cost-effective production of malonate from GHGs (greenhouse gases)-CH₄ and CO₂, can be used to store away GHGs from the atmosphere for longer term and substitute petroleum dependence for malonate production. CH₄ is a low-cost feedstock compared to sugar when used for malonate production. As government and venture capital interests are now drawn to climate crisis, investments and new companies in GHGs capture, storage and utilization technologies are a promising future.

Other objects, features, and advantages of the present invention will be apparent to one of skill in the art from the following detailed description and figures.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1. Sources of methane.

FIG. 2. Established method of DNA transfer-conjugation.

FIG. 3. Malonic acid derivatives, and reaction to produce malonic acid.

FIG. 4. Electroporation protocol optimization.

FIG. 5. Observation-autoclaved water, 1.8 kV, cuvette 1 mm, recovery overnight.

FIG. 6. DNA methylation plays a significant role in transformation efficiency.

FIG. 7. Two strains (pMTV64, pMTV65) worked similar to Top10 F′ in panel A in DASS and panel B in WT.

FIG. 8. Inducible PMB (methyl benzoate) promoter and constitutive promoter sucrose phosphate synthase P (sps) fluorescence with GFP.

FIG. 9. Schematic of CH₄ to malonate formation with introduction of a recombinant malonyl-CoA hydrolases pathway.

FIG. 10. Cloning and activity of Ach-H.

FIG. 11. Workflow of malonate detection from DASS transformants.

FIG. 12. Detection of malonate in strain DASS with heterologous hydrolases. No malonate is detected in strain DASS. All 4 screened enzymes from round 1 were put on a plasmid and introduced to DASS, and malonate was detected. POA8Y8EC has the highest titer for malonate (12.3 mg/L) at the 48-hour sample compared to all other samples and it was selected for the further engineering (round 2). Malonate was produced solely from methane. “UD” is “undetected”.

FIG. 13 shows malonate titer strain comparison: WT vs DASS. U.S. Patent Application Publication No. 2023/0399666 disclosed that strain DASS produced more fatty acids compared to the corresponding WT (wild type). The DASS strain had higher malonyl-CoA pool than WT. Using malonyl-CoA to malonate conversion pathway with our screened enzyme. The DASS strain with POA8Y8_{E.c.} has 1.5 times higher titer of malonate than WT strain, solely from methane. The same titer from methanol should also be achieved.

FIG. 14 shows enzyme activity for POA8Y8_{EC} variants on Malonyl-CoA substrate. Round 2. Malonyl-CoA (substrate) specificity increased by our mutations in WT protein (WT-wild type or non-engineered POA8Y8 protein) result spanning from WT to I116. Mutations P49F, G51F and I116F had the highest specific activity to Malonyl-CoA. Round 3 strategy: further enzyme engineering was performed by creating double and triple mutations of highest activity reported by three mutants. Double mutant P49F+G51F and triple enzyme mutant P49F+G51F+1116F reported ˜1.5 and ˜2 fold highest enzyme activity to malonyl-CoA, respectively.

FIG. 15 shows malonic acid titer of POA8Y8_{EC} mutants. All of the rationally designed mutants of POA8Y8_{Ec} had higher malonic acid titer than the WT or unmutated protein POA8Y8_{Ec}. The triple mutant P49FG51FI116F has an about 2.8-fold increase in malonic acid titer (about 32 mg/L) than that of WT (POA8Y8_{Ec}; 12.3 mg/L). Malonic acid was produced solely by utilizing methane and CO₂ generated as a byproduct of cellular methane metabolism. The same pathway works as efficiently with methanol as the substrate.

### DETAILED DESCRIPTION OF THE INVENTION

Before the invention is described in detail, it is to be understood that, unless otherwise indicated, this invention is not limited to particular sequences, expression vectors, enzymes, host cells, microorganisms, or processes, as such may vary. It is also to be understood that the terminology used herein is for purposes of describing particular embodiments only, and is not intended to be limiting.

As used in the specification and the appended claims, the singular forms “a,” “an,” and “the” include plural referents unless the context clearly dictates otherwise. Thus, for example, reference to an “expression vector” includes a single expression vector as well as a plurality of expression vectors, either the same (e.g., the same operon) or different; reference to “cell” includes a single cell as well as a plurality of cells; and the like.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

The term “about” as used herein means a value that includes 10% less and 10% more than the value referred to.

The terms “host cell” and “host microorganism” are used interchangeably herein to refer to a living biological cell, such as a microorganism, that can be transformed via insertion of an expression vector. Thus, a host organism or cell as described herein may be a prokaryotic organism (e.g., an organism of the kingdom Eubacteria) or a eukaryotic cell. As will be appreciated by one of ordinary skill in the art, a prokaryotic cell lacks a membrane-bound nucleus, while a eukaryotic cell has a membrane-bound nucleus.

The term “heterologous DNA” as used herein refers to a polymer of nucleic acids wherein at least one of the following is true: (a) the sequence of nucleic acids is foreign to (i.e., not naturally found in) a given host cell; (b) the sequence may be naturally found in a given host cell, but in an unnatural (e.g., greater than expected) amount; or (c) the sequence of nucleic acids comprises two or more subsequences that are not found in the same relationship to each other in nature. The term “heterologous” as used herein refers to a structure or molecule wherein at least one of the following is true: (a) the structure or molecule is foreign to (i.e., not naturally found in) a given host cell; or (b) the structure or molecule may be naturally found in a given host cell, but in an unnatural (e.g., greater than expected) amount. For example, regarding instance (c), a heterologous nucleic acid sequence that is recombinantly produced will have two or more sequences from unrelated genes arranged to make a new functional nucleic acid. Specifically, the present invention describes the introduction of an expression vector into a host cell, wherein the expression vector contains a nucleic acid sequence coding for an enzyme that is not normally found in a host cell. With reference to the host cell's genome, then, the nucleic acid sequence that codes for the enzyme is heterologous.

The terms “expression vector” or “vector” refer to a compound and/or composition that transduces, transforms, or infects a host cell, thereby causing the cell to express nucleic acids and/or proteins other than those native to the cell, or in a manner not native to the cell. An “expression vector” contains a sequence of nucleic acids (ordinarily RNA or DNA) to be expressed by the host cell. Optionally, the expression vector also comprises materials to aid in achieving entry of the nucleic acid into the host cell, such as a virus, liposome, protein coating, or the like. The expression vectors contemplated for use in the present invention include those into which a nucleic acid sequence can be inserted, along with any preferred or required operational elements. Further, the expression vector must be one that can be transferred into a host cell and replicated therein. Preferred expression vectors are plasmids, particularly those with restriction sites that have been well documented and that contain the operational elements preferred or required for transcription of the nucleic acid sequence. Such plasmids, as well as other expression vectors, are well known to those of ordinary skill in the art.

The term “transduce” as used herein refers to the transfer of a sequence of nucleic acids into a host cell or cell. Only when the sequence of nucleic acids becomes stably replicated by the cell does the host cell or cell become “transformed.” As will be appreciated by those of ordinary skill in the art, “transformation” may take place either by incorporation of the sequence of nucleic acids into the cellular genome, i.e., chromosomal integration, or by extrachromosomal integration. In contrast, an expression vector, e.g., a virus, is “infective” when it transduces a host cell, replicates, and (without the benefit of any complementary virus or vector) spreads progeny expression vectors, e.g., viruses, of the same type as the original transducing expression vector to other microorganisms, wherein the progeny expression vectors possess the same ability to reproduce.

As used herein, the terms “nucleic acid sequence,” “sequence of nucleic acids,” and variations thereof shall be generic to polydeoxyribonucleotides (containing 2-deoxy-D-ribose), to polyribonucleotides (containing D-ribose), to any other type of polynucleotide that is an N-glycoside of a purine or pyrimidine base, and to other polymers containing nonnucleotidic backbones, provided that the polymers contain nucleobases in a configuration that allows for base pairing and base stacking, as found in DNA and RNA. Thus, these terms include known types of nucleic acid sequence modifications, for example, substitution of one or more of the naturally occurring nucleotides with an analog; intemucleotide modifications, such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., arninoalklyphosphoramidates, aminoalkylphosphotriesters); those containing pendant moieties, such as, for example, proteins (including nucleases, toxins, antibodies, signal peptides, poly-L-lysine, etc.); those with intercalators (e.g., acridine, psoralen, etc.); and those containing chelators (e.g., metals, radioactive metals, boron, oxidative metals, etc.). As used herein, the symbols for nucleotides and polynucleotides are those recommended by the IUPAC-IUB Commission of Biochemical Nomenclature (Biochem. 9:4022, 1970).

The term “operably linked” refers to a functional linkage between a nucleic acid expression control sequence (such as a promoter) and a second nucleic acid sequence, wherein the expression control sequence directs transcription of the nucleic acid corresponding to the second sequence.

### Enzymes, and Nucleic Acids Encoding Thereof

A homologous enzyme is an enzyme that has a polypeptide sequence that is at least 70%, 75%, 80%, 85%, 90%, 95% or 99% identical to any one of the enzymes described in this specification or in an incorporated reference. The homologous enzyme retains amino acids residues that are recognized as conserved for the enzyme. The homologous enzyme may have non-conserved amino acid residues replaced or found to be of a different amino acid, or amino acid(s) inserted or deleted, but which does not affect or has insignificant effect on the enzymatic activity of the homologous enzyme. The homologous enzyme has an enzymatic activity that is identical or essentially identical to the enzymatic activity any one of the enzymes described in this specification or in an incorporated reference. The homologous enzyme may be found in nature or be an engineered mutant thereof.

The nucleic acid constructs of the present invention comprise nucleic acid sequences encoding one or more of the subject enzymes. The nucleic acid of the subject enzymes is operably linked to promoters and optionally control sequences such that the subject enzymes are expressed in a host cell cultured under suitable conditions. The promoters and control sequences are specific for each host cell species. In some embodiments, expression vectors comprise the nucleic acid constructs. Methods for designing and making nucleic acid constructs and expression vectors are well known to those skilled in the art.

Sequences of nucleic acids encoding the subject enzymes are prepared by any suitable method known to those of ordinary skill in the art, including, for example, direct chemical synthesis or cloning. For direct chemical synthesis, formation of a polymer of nucleic acids typically involves sequential addition of 3′-blocked and 5′-blocked nucleotide monomers to the terminal 5′-hydroxyl group of a growing nucleotide chain, wherein each addition is effected by nucleophilic attack of the terminal 5′-hydroxyl group of the growing chain on the 3′-position of the added monomer, which is typically a phosphorus derivative, such as a phosphotriester, phosphoramidite, or the like. Such methodology is known to those of ordinary skill in the art and is described in the pertinent texts and literature (e.g., in Matteuci et al. (1980) Tet. Lett. 521:719; U.S. Pat. Nos. 4,500,707; 5,436,327; and 5,700,637). In addition, the desired sequences may be isolated from natural sources by splitting DNA using appropriate restriction enzymes, separating the fragments using gel electrophoresis, and thereafter, recovering the desired nucleic acid sequence from the gel via techniques known to those of ordinary skill in the art, such as utilization of polymerase chain reactions (PCR; e.g., U.S. Pat. No. 4,683,195).

Each nucleic acid sequence encoding the desired subject enzyme can be incorporated into an expression vector. Incorporation of the individual nucleic acid sequences may be accomplished through known methods that include, for example, the use of restriction enzymes (such as BamHI, EcoRI, Hhal, Xhol, Xmal, and so forth) to cleave specific sites in the expression vector, e.g., plasmid. The restriction enzyme produces single stranded ends that may be annealed to a nucleic acid sequence having, or synthesized to have, a terminus with a sequence complementary to the ends of the cleaved expression vector. Annealing is performed using an appropriate enzyme, e.g., DNA ligase. As will be appreciated by those of ordinary skill in the art, both the expression vector and the desired nucleic acid sequence are often cleaved with the same restriction enzyme, thereby assuring that the ends of the expression vector and the ends of the nucleic acid sequence are complementary to each other. In addition, DNA linkers may be used to facilitate linking of nucleic acids sequences into an expression vector.

A series of individual nucleic acid sequences can also be combined by utilizing methods that are known to those having ordinary skill in the art (e.g., U.S. Pat. No. 4,683,195).

For example, each of the desired nucleic acid sequences can be initially generated in a separate PCR. Thereafter, specific primers are designed such that the ends of the PCR products contain complementary sequences. When the PCR products are mixed, denatured, and reannealed, the strands having the matching sequences at their 3′ ends overlap and can act as primers for each other Extension of this overlap by DNA polymerase produces a molecule in which the original sequences are “spliced” together. In this way, a series of individual nucleic acid sequences may be “spliced” together and subsequently transduced into a host cell simultaneously. Thus, expression of each of the plurality of nucleic acid sequences is effected.

Individual nucleic acid sequences, or “spliced” nucleic acid sequences, are then incorporated into an expression vector. The invention is not limited with respect to the process by which the nucleic acid sequence is incorporated into the expression vector. Those of ordinary skill in the art are familiar with the necessary steps for incorporating a nucleic acid sequence into an expression vector. A typical expression vector contains the desired nucleic acid sequence preceded by one or more regulatory regions, along with a ribosome binding site, e.g., a nucleotide sequence that is 3-9 nucleotides in length and located 3-11 nucleotides upstream of the initiation codon in E. coli. See Shine et al. (1975) Nature 254:34 and Steitz, in Biological Regulation and Development: Gene Expression (ed. R. F. Goldberger), vol. 1, p. 349, 1979, Plenum Publishing, N.Y.

Regulatory regions include, for example, those regions that contain a promoter and an operator. A promoter is operably linked to the desired nucleic acid sequence, thereby initiating transcription of the nucleic acid sequence via an RNA polymerase enzyme. An operator is a sequence of nucleic acids adjacent to the promoter, which contains a protein-binding domain where a repressor protein can bind. In the absence of a repressor protein, transcription initiates through the promoter. When present, the repressor protein specific to the protein-binding domain of the operator binds to the operator, thereby inhibiting transcription. In this way, control of transcription is accomplished, based upon the particular regulatory regions used and the presence or absence of the corresponding repressor protein. An example includes lactose promoters (LacI repressor protein changes conformation when contacted with lactose, thereby preventing the LacI repressor protein from binding to the operator). Another example is the tac promoter. (See deBoer et al. (1983) Proc. Natl. Acad. Sci. USA, 80:21-25.) As will be appreciated by those of ordinary skill in the art, these and other expression vectors may be used in the present invention, and the invention is not limited in this respect.

Although any suitable expression vector may be used to incorporate the desired sequences, readily available expression vectors include, without limitation: plasmids, such as pSC101, pBR322, pBBRIMCS-3, pUR, pEX, pMR100, pCR4, pBAD24, pUC19; bacteriophages, such as M13 phage and λ phage. Of course, such expression vectors may only be suitable for particular host cells. One of ordinary skill in the art, however, can readily determine through routine experimentation whether any particular expression vector is suited for any given host cell. For example, the expression vector can be introduced into the host cell, which is then monitored for viability and expression of the sequences contained in the vector. In addition, reference may be made to the relevant texts and literature, which describe expression vectors and their suitability to any particular host cell.

The expression vectors of the invention must be introduced or transferred into the host cell. Such methods for transferring the expression vectors into host cells are well known to those of ordinary skill in the art. For example, one method for transforming E. coli with an expression vector involves a calcium chloride treatment wherein the expression vector is introduced via a calcium precipitate. Other salts, e.g., calcium phosphate, may also be used following a similar procedure. In addition, electroporation (i.e., the application of current to increase the permeability of cells to nucleic acid sequences) may be used to transfect the host cell. Also, microinjection of the nucleic acid sequencers) provides the ability to transfect host cell. Other means, such as lipid complexes, liposomes, and dendrimers, may also be employed. Those of ordinary skill in the art can transfect a host cell with a desired sequence using these or other methods.

For identifying a transfected host cell, a variety of methods are available. For example, a culture of potentially transfected host cells may be separated, using a suitable dilution, into individual cells and thereafter individually grown and tested for expression of the desired nucleic acid sequence. In addition, when plasmids are used, an often-used practice involves the selection of cells based upon antimicrobial resistance that has been conferred by genes intentionally contained within the expression vector, such as the amp, gpt, neo, and hyg genes.

In some embodiments, the host cells are genetically modified in that heterologous nucleic acid have been introduced into the host cells, and as such the genetically modified host cells do not occur in nature. The suitable host cell is one capable of expressing a nucleic acid construct encoding one or more enzymes described herein. The gene(s) encoding the enzyme(s) may be heterologous to the host cell or the gene may be native to the host cell but is operatively linked to a heterologous promoter and one or more control regions which result in a higher expression of the gene in the host cell.

The enzyme can be native or heterologous to the host cell. Where the enzyme is native to the host cell, the host cell is genetically modified to modulate expression of the enzyme. This modification can involve the modification of the chromosomal gene encoding the enzyme in the host cell or a nucleic acid construct encoding the gene of the enzyme is introduced into the host cell. One of the effects of the modification is the expression of the enzyme is modulated in the host cell, such as the increased expression of the enzyme in the host cell as compared to the expression of the enzyme in an unmodified host cell.

The amino acid sequence of E. coli POA8Y8 is:

(SEQ ID NO: 1)

MIWKRHLTLD ELNATSDNTM VAHLGIVYTR LGDDVLEAEM
PVDTRTHQPF GLLHGGASAA LAETLGSMAG EMMIRDGQCV
VGTELNATHH RPVSEGKVRG VCQPLHLGRQ NQSWEIVVED
EQGRRCCTCR LGTAVLG

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The invention having been described, the following examples are offered to illustrate the subject invention by way of illustration, not by way of limitation.

### Example 1

### Development of Genetic Tools and a Recombinant Malonate Pathway in Methylotuvimicrobium alcaliphilum

Methane is one of the most potent greenhouse gases of the atmosphere and an economical feedstock for biological conversion of methane to biochemicals and bioproducts. Methanotrophs are selective microbes that grow on methane and can be engineered to produce targeted chemicals. However, the efficiency and efforts in engineering these hosts needs development and much exploration. In this study, 1) we have optimized electroporation mode of transformation for plasmids in to a methanotrophic bacteria, Methylotuvimicrobium alcaliphilum, significantly bringing down time for DNA transfer; 2) Evaluated promoters (constitutive and inducible) for driving gene expression using GFP (green fluorescent protein) reporter; 3) Screened and established a novel malonate biosynthesis route in the methanotrophic host, M. alcaliphilum. Malonic acid, one of the top 30 biochemicals listed by the U.S. Department of Energy, is an attractive platform chemical. Towards methanotroph engineering efforts for malonate, we screened 4 putative candidate malonyl-CoA hydrolases and achieved 24 mg/L malonate titer with the best candidate (E. coli POA8Y8). Genetic tools and recombinant pathway developed in this study can be expanded to other methanotrophs for targeted biochemical synthesis.

Bioconversion provides identical performance without the incumbent toxic production process. Methane is ˜25 times cheaper than sugar (glucose) and available as renewable and non-renewable Carbon source.

The method of the present invention comprises the one step conversion of malonyl-CoA to malonate by a potential malonyl-CoA hydrolases: this pathway uses 2 mol CH₄ and 1 mol CO₂ (generated as byproduct of cellular oxidation of CH₄). As such, the method is a process that has a reduced carbon footprint, reduced carbon emission, and net negative GHG.

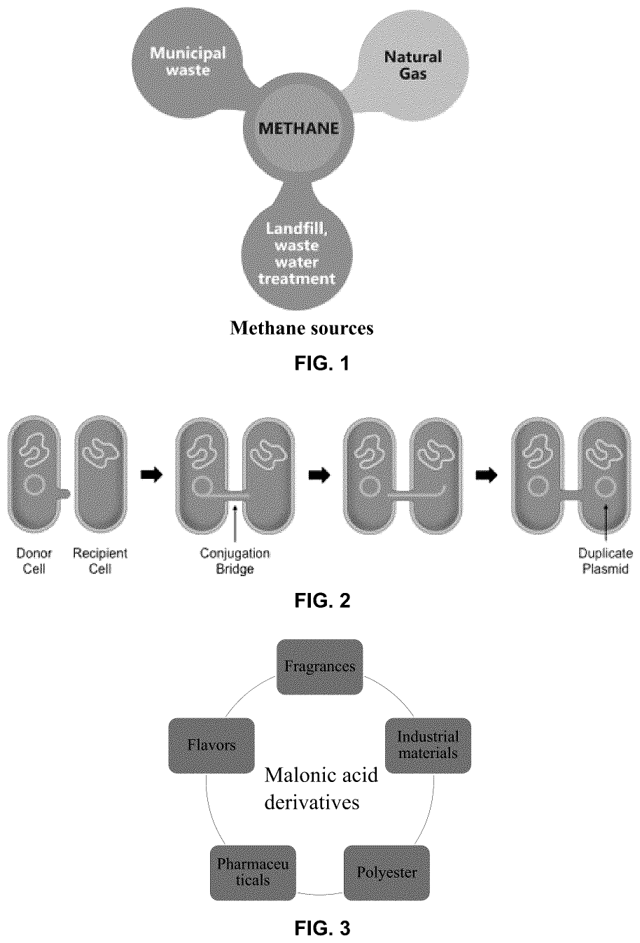

The method uses methane as feed stock to produce malonate. Currently methane is used primarily as fuel to generate heat and light. See FIG. 1. Biotechnologies to efficiently convert methane to biochemicals can bring new sustainable solutions to several industries with large environmental footprints.

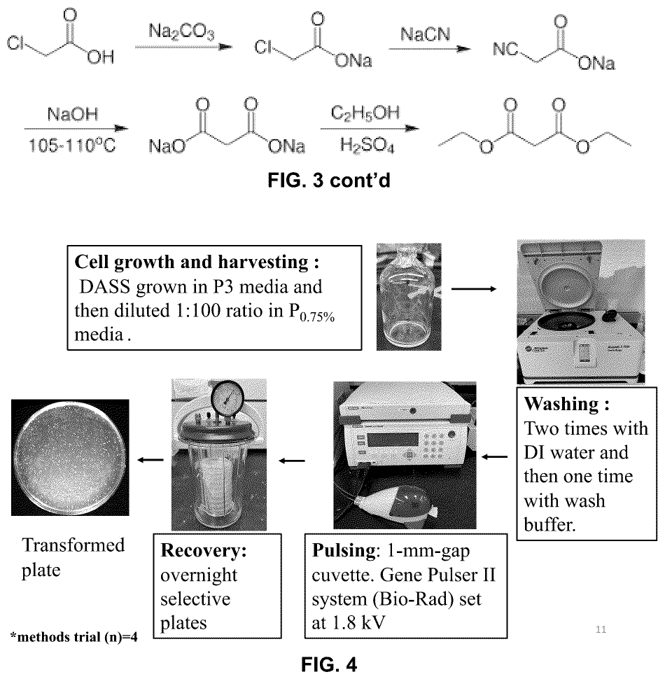

The electroporation conjugation method is time consuming, taking about 4-5 weeks. See FIG. 2. (Established method of DNA transfer-conjugation.) Electroporation can bring down the time and efforts of DNA transformation by half. Malonic acid is a molecule chosen to be a target molecule to be produced. Currently it is produced using the reaction shown in FIG. 3. The global market for malonate is about $14 billion/year.

### Results

The strategy used comprised: (1) One step conversion of malonyl-CoA to malonate. (2) Fix two GHG (greenhouse gases) CH₄ and CO₂ in the process. (3) Identify and screen potential malonyl-CoA hydrolases that catalyze the reaction. (4) Protein engineering and expression in strain DASS (strain with high flux to fatty acids and malonyl-CoA).

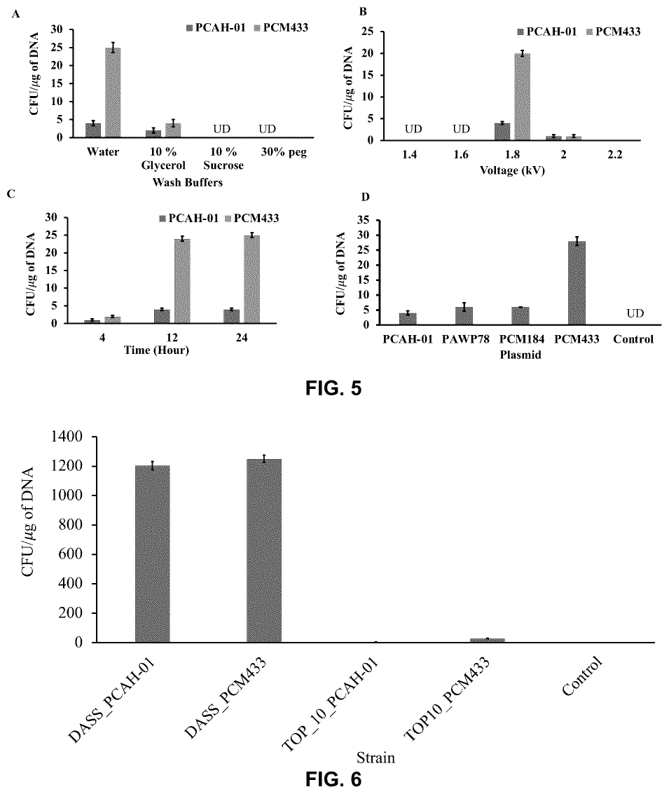

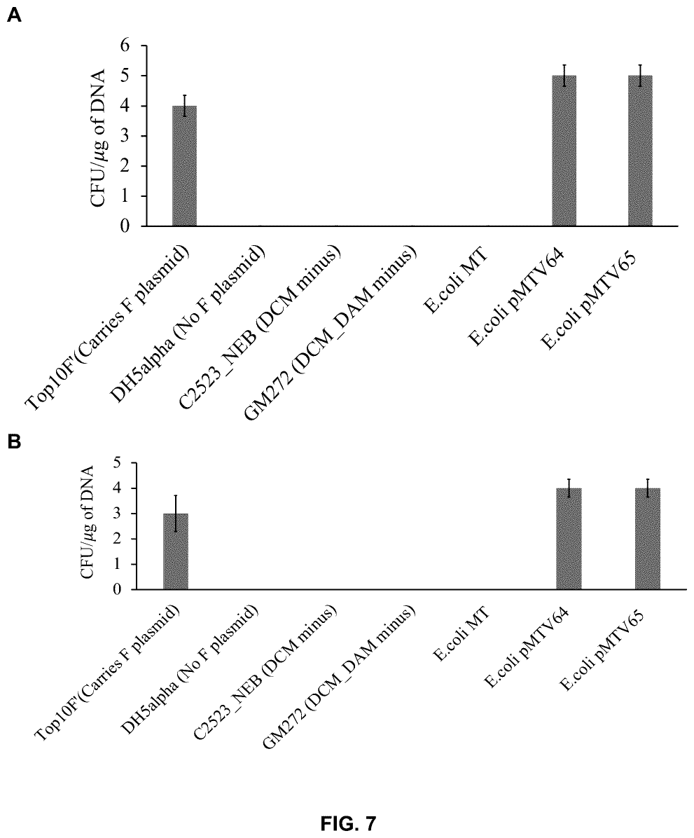

The method described herein comprises a method for optimized protocol for electroporation. See FIG. 4 (Electroporation protocol optimization.) FIG. 5 shows observation for electroporation results using autoclaved water, 1.8 kV, cuvette 1 mm, recovery overnight. FIG. 6 shows DNA methylation plays a significant role in transformation efficiency. FIG. 7A and FIG. 7 B show two strains (pMTV64, pMTV65) worked similar to Top10 F′ in 7A in DASS and 7B in WT (Courtesy of Dr. Adam Guss, Oak Ridge National Laboratory, Oak Ridge, TN). “WT” is WT: Methylotuvimicrobium alcaliphilum DSM19304.

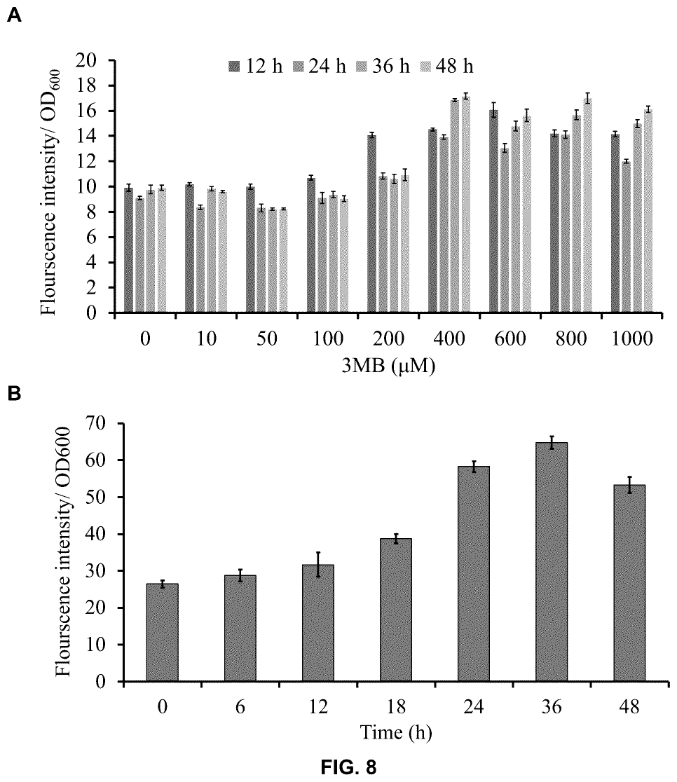

Promoter screening and engineering results are shown herein. FIG. 8 shows inducible PMB (methyl benzoate) promoter and constitutive promoter sucrose phosphate synthase P (sps) fluorescence with GFP.

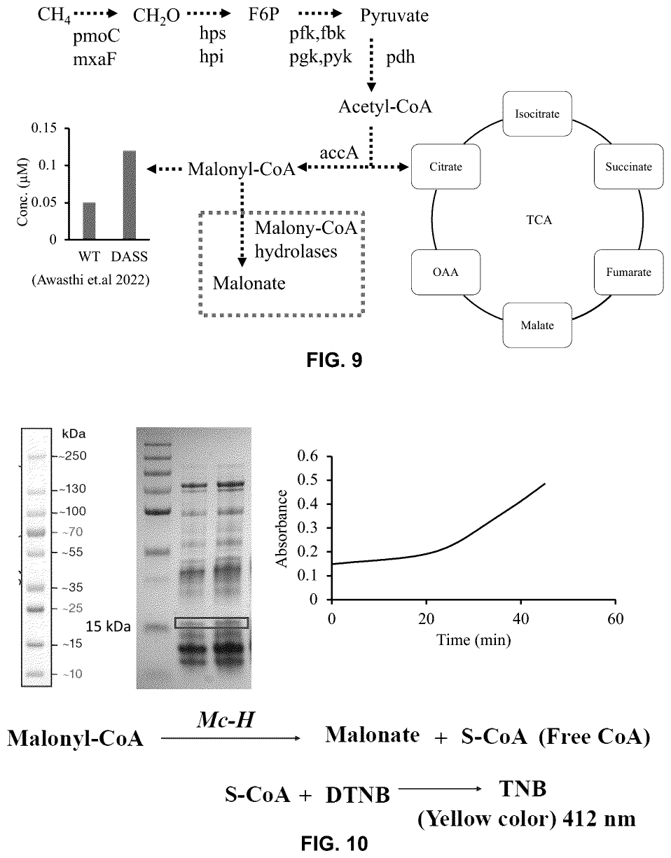

Engineer a new pathway for malonic acid production. FIG. 9 shows schematic of CH₄ to malonate formation with introduction of a recombinant malonyl-CoA hydrolases pathway. FIG. 9 shows a metabolic pathway for producing malonate from methane and carbon dioxide, and the enzymes needed for the pathway.

Malonyl-CoA hydrolase candidates were screened. Short chain acyl-CoA hydrolases are pre-screened for malonyl-CoA hydrolase activity. Malonate was not detected in strain DASS. Therefore, recombinant enzymes are required for malonate production by this strain. Through literature review 4 short chain acyl-CoA hydrolases were identified for pre-screened for malonyl-CoA hydrolase activity. Table 2 shows the published kinetic parameters, origin of the enzyme, and the evaluated malonate titer after incorporating them in strain DASS (plasmid based).

TABLE 2
Selection of acyl-CoA hydrolases (Ac-H) as a potential malonyl-CoA hydrolase (Mc-H).

| | | | | | Malonate |
|---|---|---|---|---|---|
| | | | | Cloned | titer |
| | | | | and | achieved |
| | | Characterization | Kinetic parameter | expressed | in DASS |
| Candidate | Organism | on Acyl-CoA | (Spe. Activity, | in strain | (carrying |
| Enzymes | (reference) | thioesters | Kₘ, K_{cat}) | DASS | enzyme) |
| YBGC | Haenophilus influenzae Rd | Propionyl-CoA | K_{cat}: 0.44 ± .04/s | Yes | 13.3 |
| | KW20 (webpage for: | | Kₘ: 11 ± 1 mM | | mg/L |
| | doi.org/10.1016/S0014- | | | | |
| | 5793(02)02533-4) | | | | |
| YCIA | Haenophilus influenzae Rd | Malonyl-CoA, | K_{cat}: 3.3 ± .2/s | Yes | 12.7 |
| | KW20 (webpages for: | Propionyl-CoA | Kₘ: 140 ± 20 μM | | mg/L |
| | doi.org/10.1021/bi702334h, | | K_{cat}/Kₘ: | | |
| | doi.org/10.1021/bi702336d) | | 2.4 × 10⁴/M | | |
| P0A8Y8 | Escherichia coli (strain | Propionyl-CoA | Kₘ: 400 ± 20 μM | Yes | 13.8 |
| | K12) (webpage for: | | | | mg/L |
| | doi.org/10.1021/bi702334h) | | | | |
| P0A8Z3 | Escherichia coli (strain | Malonyl-CoA | No record | Yes | 12.9 |
| | K12) (webpage for: | | | | mg/L |
| | doi.org/10.1021/bi702334h) | | | | |

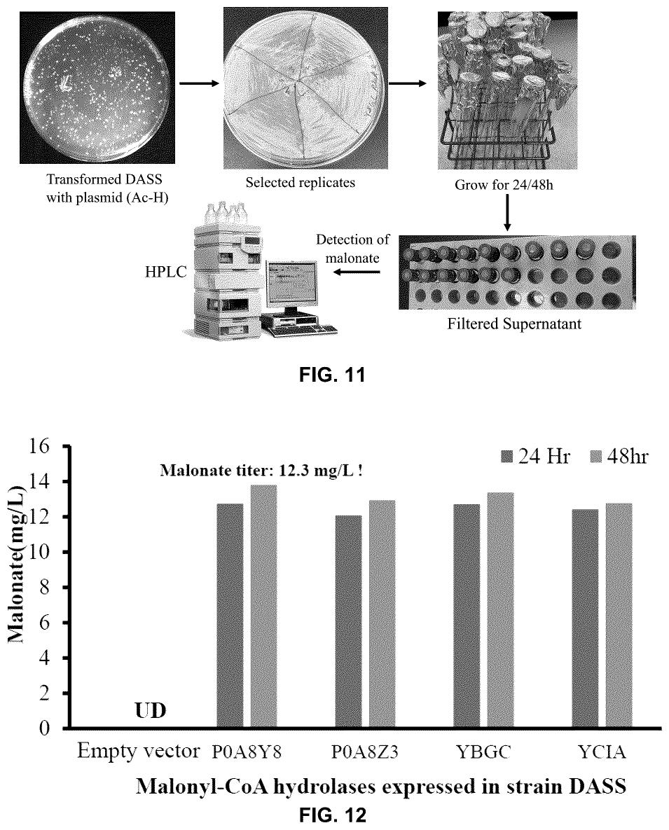

Cloning and activity confirmation of Ac-H in E. coli. FIG. 10 shows cloning and activity of Ach-H. Next, 4 Ach-H enzymes were cloned in pDA21 under Pₛₚₛ-Ac-H and were electroporated into strain DASS using the optimized protocol described herein. FIG. 11 shows the workflow of malonate detection from DASS transformants. Malonate is detected in DASS with Ach-H colonies. FIG. 12 shows the malonate titer of Ach-H compared with DASS. POA8Y8_{EC} has the highest titer observed compared to all other Ac-Hs and, is selected for the further engineering. Table 3 shows the POA8Y8_{EC} Selected candidate for protein engineering. Selected amino acid locations for mutation of POA8Y8 from E. coli for enzyme engineering to enhance specificity to substrate, malonyl-CoA and eventually improve malonate titer and yield.

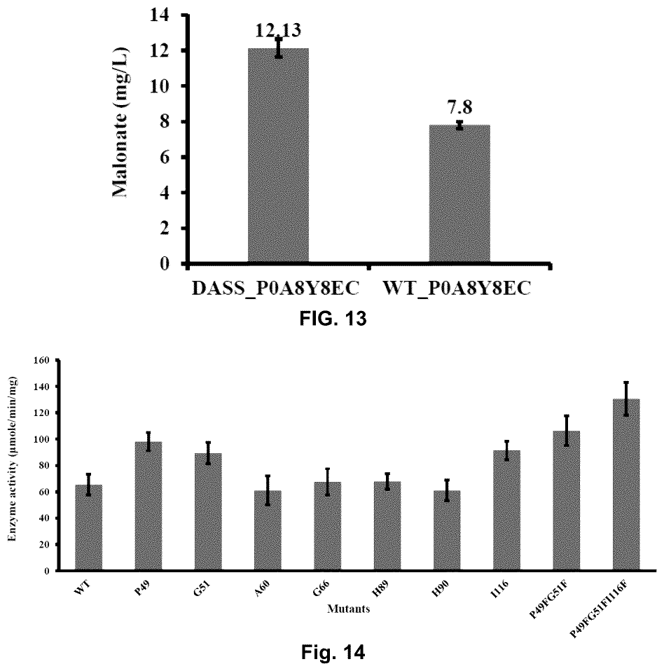

FIG. 13 shows malonate titer strain comparison: WT vs DASS. U.S. Patent Application Publication No. 2023/0399666 disclosed that strain DASS produced more fatty acids compared to the corresponding WT (wild type). The DASS strain had higher malonyl-CoA pool than WT. Using malonyl-CoA to malonate conversion pathway with our screened enzyme. The DASS strain with POA8Y8_{E.c}. has 1.5 times higher titer of malonate than WT strain, solely from methane. The same titer from methanol should also be achieved.

E. coli POA8Y8 (POA8Y8_{Ec}) has published active sites (catalytic residues) at Q48 and H54 (chain one), and E63, F64, S67, and M68 (chain two). The following amino acid residues were selected for mutation: P49, G51, A60, G66, H89, H90, P92, and I116.

TABLE 3
P0A8Y8_{EC} Selected candidate for protein engineering.

| Selected | Mutation | |
|---|---|---|
| residues | designed | Rational |
| P49 | P49F | Increased steric and |
| G51 | G51F | hydrophobicity* |
| A60 | A60F | |
| G66 | G66F | |
| H89 | H89F | |
| H90 | H90F | |
| P92 | P92F | |
| I116 | I116F | |

*Selected amino acids have been mutated to phenylalanine

Selected amino acid residues have been mutated to phenylalanine to increase steric hindrance and hydrophobicity approach.

FIG. 14 shows enzyme activity for POA8Y8_{EC} variants on Malonyl-CoA substrate. Round 2. Malonyl-CoA (substrate) specificity increased by our mutations in WT protein (WT-wild type or non-engineered POA8Y8 protein) result spanning from WT to 1116. Mutations P49F, G51F and I116F had the highest specific activity to Malonyl-CoA. Round 3 strategy: further enzyme engineering was performed by creating double and triple mutations of highest activity reported by three mutants. Double mutant P49F+G51F and triple enzyme mutant P49F+G51F+1116F reported ˜1.5 and ˜2 fold highest enzyme activity to malonyl-CoA, respectively.

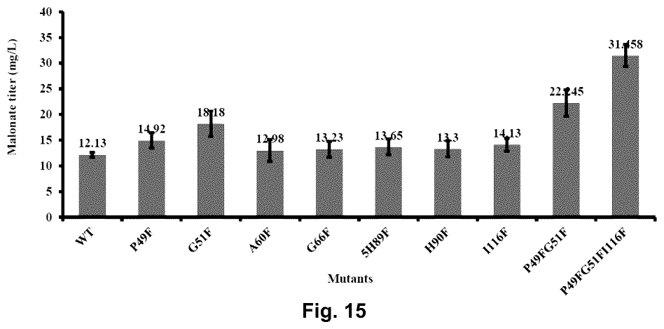

FIG. 15 shows malonic acid titer of POA8Y8_{EC} mutants. All of the rationally designed mutants of POA8Y8_{Ec} had higher malonic acid titer than the WT or unmutated protein POA8Y8_{Ec}. The triple mutant P49FG51FI116F has an about 2.8-fold increase in malonic acid titer (about 32 mg/L) than that of WT (POA8Y8_{Ec}; 12.3 mg/L). Malonic acid was produced solely by utilizing methane and CO₂ generated as a byproduct of cellular methane metabolism. The same pathway works as efficiently with methanol as the substrate.

### CONCLUSION

Established a robust electroporation protocol for DNA transformation. Screened tightly controlled inducible promoter (PMB) and Pₛₚₛ constitutive promoter. Identified and screened four short chain Acyl-CoA hydrolases with malonyl-CoA activity. In the process of further enzyme engineering of the best candidate-POA8Y8_{EC}.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

We claim:

1. A genetically modified host cell is Methylomicrobium species cell comprising a heterologous acyl-CoA hydrolase having the enzymativ activity to convert malonyl-CoA into malonate.

2. The genetically modified host cell of claim 1, wherein the Methylomicrobium species cell is Methylomicrobium alcaliphilum cell.

3. The genetically modified host cell of claim 2, wherein the Methylomicrobium alcaliphilum cell is Methylomicrobium alcaliphilum strain DSM19304.

4. The genetically modified host cell of claim 3, wherein the Methylomicrobium alcaliphilum strain DSM19304 is DASS strain.

5. The genetically modified host cell of claim 1, wherein the acyl-CoA hydrolase is a short-chain acyl-CoA hydrolase.

6. The genetically modified host cell of claim 5, wherein the acyl-CoA hydrolase is a short-chain acyl-CoA hydrolase.

7. The genetically modified host cell of claim 6, wherein the acyl-CoA hydrolase is an Escherichia coli (E. coli) POA8Z3, E. coli POA8Y8, Haemophilus influenzae Rd KW20 YBGC, H. influenzae YciA, or Mesocricetus auratus Acot9, or a wild-type or homologous enzyme.

8. The genetically modified host cell of claim 7, wherein the acyl-CoA hydrolase comprises at least one or more, or all, of the conserved amino acid residues compared to any two of any of the following: E. coli POA8Z8, Salmonella typhimurium proofreading thiosterase EntH, Salmonella schwarzengrund proofreading thiosterase EntH, E. coli proofreading thiosterase EntH, Citrobacter koseri proofreading thiosterase EntH, Salmonella paratyphi A proofreading thiosterase EntH, Salmonella paratyphi B proofreading thiosterase EntH, Shigella flexneri proofreading thiosterase EntH, Shigella dysenteriae proofreading thiosterase EntH, and Shigella sonnei proofreading thiosterase EntH. (webpage for: uniprot.org/uniprotkb/POA8Y8/entry).

9. The genetically modified host cell of claim 8, wherein the acyl-CoA hydrolase comprises one or more of the following amino acid residues corresponding to SEQ ID NO:1 is substituted with a different amino acid: P49, G51, A60, G66, H89, H90, P92, and I116.

10. The genetically modified host cell of claim 9, wherein the amino acid residue(s) is substituted with an amino acid residue with a bulky sidechain.

11. The genetically modified host cell of claim 10, wherein the amino acid residue(s) is substituted with a phenylalanine.

12. A method for producing a malonate, the method comprising: (a) providing a genetically modified host cell of claim 1, capable of expression in the host cell in a growth or culture medium comprising methane; (b) growing or culturing the host cell such that a malonate is produced; and (c) optionally recovering the malonate from the host cell or from the growth or culture medium.
